(19) United States
(12) Patent Application Publication
(10) Pub. No.: US 2004/0248238 A1
(43) Pub. Date: Dec. 9, 2004

(54) Method for increasing the solubility, expression rate and the acitivity of proteins during recombinant production

(76) Inventors: Manfred Watzele, Weilheim (DE); Regina Schweizer, Polling (DE); Cordula Nemetz, Wolfratshausen (DE); Robin Steigerwald, Muenchen (DE); Thomas Emrich, Iffeldorf (DE); Katrin Zaiss, Muenchen (DE); Erhard Fernholz, Weilheim (DE); Baerbel Walckhoff, Tutzing (DE); Hans Joachim Schoenfeld, Freiburg i. Breisgau (DE); Birgit Offen, Muenchen (DE)

Correspondence Address: Marilyn L Amick, Roche Diagnostics Operations Inc, Building D, 9115 Hague Road, Indianapolis, IN, 46250, US

(21) Appl. No.: 10/489,941

(22) PCT Filed: Sep. 14, 2002
(86) PCT No.: PCT/EP02/10329

(30) Foreign Application Priority Data
Sep. 17, 2001 (DE) 10145694.8

(51) Int. Cl. C12P 21/06; A61K 38/00

(52) U.S. Cl. 435/68.1; 514/2

(57) ABSTRACT

The present invention concerns a method for producing a lysate containing helper proteins in which a strain which is suitable for obtaining in vitro translation lysates is transformed using a vector containing one or more genes coding for one or more helper proteins, wherein the helper proteins are expressed in this strain and the lysate containing helper proteins is obtained from these strains. The present invention also concerns a lysate containing helper proteins that can be obtained by the method according to the invention, blends of these lysates and the use of these lysates and blends in in vitro translation systems.

### FIELD OF THE INVENTION

The present invention concerns a method for producing a lysate containing helper proteins in which a strain which is suitable for obtaining in vitro translation lysates is transformed using a vector containing one or more genes coding for one or more helper proteins, wherein the helper proteins are expressed in this strain and the lysate containing helper proteins is obtained from these strains. The present invention also concerns a lysate containing helper proteins that can be obtained by the method according to the invention, blends of these lysates and the use of these lysates and blends in in vitro translation systems.

### BACKGROUND OF THE INVENTION

The addition of highly purified helper proteins has already been described in the prior art. Thus the application WO 94/24303 describes the use of DnaJ, DnaK, GrpE, GroEL and GroES for activating a protein synthesized in vitro. It describes a cell-free extract which is substantially free of protein-degrading and DNA-degrading enzymes and incubation in an in vitro transcription/translation medium which contains helper proteins.

In WO 94/24303 as well as in Kudlicki et al. (1995), J. Bacteriol. 177, 5517 and Kudlicki et al. (1996), J. Biol. Chem. 271, 31160 an isolated complex of ribosomes and a peptide/protein is redissolved by adding helper proteins and ATP which thus releases the active protein.

Ryabova et al. (1997), Nature Biotechnology 15, 79 describe the use of DnaJ, DnaK, GrpE, GroEL and GroES acting together with protein disulfide isomerase in in vitro translation using an E. coli lysate. The addition of DnaJ, DnaK, GrpE alone increased the solubility of a disulfide-containing protein whereas the addition of protein disulfide isomerase increased the activity.

Merk et al. (1999), J. Biochem. 125, 328 describe the use of DnaJ, DnaK, GrpE, GroEL and GroES acting together with protein disulfide isomerase in coupled or linked in vitro transcription/translation using an E. coli ribosomal fraction. The addition of helper proteins increased the solubility and activity of the proteins.

Fedorof & Baldwin (1998) Meth. Enzymol. 290, 1 list helper proteins in various cell-free extracts of conventional in vitro transcription/translation preparations from E. coli, rabbit reticulocytes and wheat germ.

The importance of various helper proteins in cotranslational protein folding and the (above-mentioned) examples in in vitro protein synthesis are summarized in Fedorof & Baldwin (1997), J. Biol. Chem. 272, 5.

Hence in the prior art highly purified helper proteins are added or use is made of helper proteins that are present in the lysate. The addition of purified helper proteins is uneconomical, whereas the helper proteins present in the lysates are in general not sufficient to adequately protect proteins from aggregation and misfolding.

EP 0885967 A2 describe the coexpression of DnaJ, DnaK, GrpE helper proteins in a cellular expression system for improving protein folding.

However, the coexpression of helper proteins is disadvantageous since the synthesis potential of the expression system has to be divided among other proteins in addition to the protein to be expressed.

Bachand et al. (2000) RNA, 6, 778 describe that human telomerase comprising the catalytic subunit hTERT and the associated RNA hTR was produced in an active form in vitro using a rabbit reticulocyte system and in vivo in yeast cells.

Holt et al. (1999) Genes & Development 13, 817 describe that other protein factors hsp 90 and p23 from the reticulocyte extract are necessary as helper proteins to reconstitute hTERT synthesized in vitro (rabbit reticulocyte system) with the associated RNA hTR.

However, telomerase cannot be expressed on a large scale in the cell-free rabbit reticulocyte system since this would require large amounts of lysate which are expensive to produce. Another objection is the protection of animals.

Masutomi et al. (2000), J. Biol. Chem., 275, 22568 describe the expression of hTERT in insect cells and its reconstitution with hTR that can be transcribed in vitro. However, they point out that all methods for synthesizing telomerase in bacterial expression systems have failed.

Weinrich et al. (1997) Nat. Genet. 17, 498 mention the successful synthesis of functionally active telomerase in a wheat germ transcription-translation system. However, experience shows that the wheat germ expression system is less productive and most of the translation products that are produced are incomplete due to the presence of high nuclease and protease activities.

### SUMMARY OF THE INVENTION

Hence the object was to develop a method which enables helper proteins to be provided in an optimal and economic manner for the in vitro synthesis of a protein (also referred to as target protein in the following). In particular, the addition of the helper proteins should be optimized such that the protein (target protein) synthesized in vitro is adequately protected from aggregation and misfolding.

The present object was achieved by a method for producing a lysate containing helper proteins, characterized in that

a strain which is suitable for obtaining in vitro translation lysates is transformed with a vector containing one or more genes coding for one or more helper proteins,

the helper proteins are expressed in this strain and

the lysate containing helper proteins is obtained from these strains.

This lysate according to the invention is then present during the in vitro synthesis of the target protein.

Helper proteins in the sense of the invention are proteins which increase the solubility, folding and/or activity of proteins expressed in vitro and can thus in some cases also increase their expression rate. A soluble protein in the sense of the invention means that the protein from the reaction mixture remains in the supernatant and does not sediment after a two minute centrifugation at 10,000-times gravitational acceleration (g). An increase in solubility in the sense of the invention means that a higher proportion of the protein (at least 10%) remains in solution when helper proteins are added than is the case for a preparation without the addition of helper proteins. Examples of helper proteins are so-called heat shock proteins and chaperones such as those from the DnaK or GroE system, chaperonins, protein disulfide isomerase, trigger factor and prolyl-cis-trans isomerase.

The folding helper proteins are selected from one or more of the following classes of protein: Hsp60, Hsp70, Hsp90, Hsp100 protein family, small heat shock protein family and isomerases.

Molecular chaperones are the largest group of folding-assisting proteins and, according to the invention, are understood as folding helper proteins (Gething and Sambrook, 1992; Hartl, 1996; Buchner, 1996; BeiBinger and Buchner, 1998). Since they are overexpressed under stress conditions, most molecular chaperones can also be classified in the group of heat shock proteins (Georgopoulos and Welch, 1993; Buchner, 1996), this group is also understood according to the invention as a folding helper protein.

Important folding helper proteins that are encompassed by the present invention are elucidated in more detail in the following. The group of molecular chaperones can be divided into five non-related protein classes on the basis of sequence homologies and molecular masses, the Hsp60, Hsp70, Hsp90, Hsp100 protein families and the family of small heat shock proteins (Gething and Sambrook, 1992; Hendrick and Hartl, 1993).

Hsp60

The best investigated chaperone overall is GroEL which is a member of the Hsp60 family from E. coli. The members of the Hsp6O family are also referred to as chaperonins and are divided into two groups. GroEL and its cochaperone GroES and their highly homologous relatives from other bacteria as well as from mitochondria and chloroplasts form the group of I chaperones. (Sigler et al., 1998; Fenton and Horwich, 1997). The Hsp60 proteins from the eukaryotic cytosol and from archebacteria comprise the group II chaperones (Gutsche et al., 1999). The Hsp60 proteins have a similar oligomeric structure in both groups. In the case of GroEL and the other group I chaperonins, 14 GroEL subunits associate to form a cylinder comprising two heptameric rings, whereas the heptameric ring structure in the case of the chaperonins of group II from archebacteria are usually composed of two different subunits. In contrast members of the group II chaperonins from the eukaryotic cytosol such as the CCT complex from yeast are composed of eight different subunits with an exactly defined organisation (Liou and Willison, 1997). Non-native proteins can be incorporated and bound in the central cavity of this cylinder. The cochaperone GroES also forms a heptameric ring and in this form binds to the poles of the GroEL cylinder. However, this binding of GroES limits substrate binding depending on its size (10-55 kDa; Ewalt et al., 1997). The substrate binding is regulated by ATP binding and hydrolysis.

Hsp70: In addition to the members of the Hsp60 family, Hsp70 proteins also bind to the nascent polypeptide chain (Beckman et al., 1990; Welch et al., 1997). There are usually several constitutively expressed and stress-induced members of the Hsp70 family in prokaryotic and eukaryotic cells (Vickery et al., 1997; Kawula and Lelivelt, 1994; Fink, 1997; Welch et al., 1997). In addition to protein folding directly on the ribosome, they are also involved in the translocation of proteins via cell and organelle membranes (Schatz & Doberstein, 1996). It has been shown that proteins can only be transported through the membrane in an unfolded or partially folded state (Hannavy et al., 1993). During the translocation process in organelles, it is above all members of the Hsp70 family that are involved in unfolding and stabilization on the cytosolic side as well as in refolding on the organelle side (Hauke and Schatz, 1997). The ATPase activity of Hsp70 is essential in all of these processes for the function of the protein. A characteristic of the Hsp70 system is that its activity is controlled by co-chaperones (Hsp40; DnaJ) and the equilibrium between substrate binding and release is influenced by specific modulation of the ATPase activity (Bukau and Horwich, 1998).

Hsp90: Hsp90 is one of the most strongly expressed proteins amounting to about 1% of the soluble protein in the eukaryotic cytosol (Welch and Feramisco, 1982). Members of this family mainly act in multimeric complexes where they recognize a large number of important signal transduction proteins with similar structures to the native proteins. These structures are stabilized by binding to Hsp90 and its partner proteins which facilitates the binding of ligands to the signal proteins. In this manner the substrates can adopt their active conformation (Sullivan et al., 1997; Bohen et al., 1995; Buchner, 1999).

Hsp100: Recently it has emerged that especially the Hsp100 chaperones are characterized by their ability in association with Hsp70 chaperones to redissolve aggregates that have already formed (Parsell et al., 1994; Golloubinoff et al., 1999; Mogk et al., 1999). Whereas their main function appears to be the mediation of thermotolerance (Schirmer et al., 1994; Kruger et al., 1994), some members such as ClpA and ClpB together with the protease subunit ClpP mediate the proteolytic degradation of proteins (Gottesman et al., 1997).

sHsps: The fifth class of chaperones, the small heat shock proteins (sHsps), is a very divergent family of heat shock proteins that are found in almost all organisms. The name for this family of chaperones relates to their relatively low monomeric molecular weights of 15-40 kDa. However, sHsps are usually present in the cell as highly oligomeric complexes comprising up to 50 subunits and thus molecular masses of 125 kDa to 2 MDa have been observed (Spector et al., 1971; Arrigo et al., 1988; Andreasi-Bassi et al., 1995; Ehrnsperger et al., 1997). Like the other chaperones, sHsps can suppress the aggregation of proteins in vitro (Horwitz, 1992; Jakob et al., 1993; Merck et al., 1993; Jakob and Buchner, 1994; Lee et al., 1995; Ehrnsperger et al., 1997 b). In this process sHsps bind up to one substrate molecule per subunit and are thus more efficient than the model chaperone GroEL (Jaenicke and Creighton, 1993; Ganea and Harding, 1995; Lee et al., 1997; Ehrnsperger et al., 1998a). Under stress conditions, the binding of non-native protein to sHsps prevents the irreversible aggregation of the proteins. Binding to sHsps keeps the proteins in a soluble folding-competent state. After physiological conditions have been restored, the non-native protein can be detached from the complex with sHsp by ATP-dependent chaperones such as Hsp70 and thus reactivated.

Isomerases: Suitable isomerases for the method according to the invention are for example folding catalysts from the class of peptidyl-prolyl-cis/trans isomerases and members of the disulfide isomerases.

Folding helper proteins that function in the same or a similar manner as the folding helper proteins described above are also encompassed by the present invention.

A particularly preferred variant of the method according to the invention is when the strain has been transformed with various vectors where at least one difference between the vectors is that the genes contained therein code for different helper proteins.

In this manner it is possible to produce different helper proteins that are important for the in vitro synthesis of the respective target protein in one lysate.

Furthermore it is also preferred according to the invention that the strain which is suitable for obtaining in vitro translation lysates additionally has at least one of the following properties: low content or deficiency of RNAse, low content or deficiency of exonuclease, low content or deficiency of protease.

One embodiment of the invention comprises the method according to the invention where the lysate is obtained in such a manner that, in addition to the helper proteins, the lysate contains all components that are necessary for an in vitro translation or for an in vitro transcription/translation of a target protein. At least the following components are required for an in vitro translation or for an in vitro transcription/translation:

ribosomes

aminoacyl tRNA synthases

initiation factors

elongation factors

termination factors

enzymes that are required to regenerate ATP, GTP, UTP and CTP starting from an added primary energy donor. Such primary energy donors are for example acetyl phosphate, creatine phosphate, phosphoenolpyruvate, pyruvate, glucose or other possible substrates known to a person skilled in the art which can be directly converted or converted by means of several enzyme-catalysed intermediate steps such that molecules with an energy-rich phosphate bond are formed which can then transfer this phosphate group to a nucleotide monophosphate or nucleotide diphosphate.

Hence the invention also concerns a lysate containing helper proteins, this lysate being obtainable by the method according to the invention. In principle other methods are also conceivable which can be used to obtain the lysate according to the invention e.g. methods in which the promoters of the helper protein genes that occur naturally in the strains are modified such that a larger amount of helper protein is formed. Another method is to transform a strain with a piece of DNA which contains the encoded helper protein and which is integrated once or several times into the genome of the strain in order to be then co-amplified by this strain during cell division. Any lysate which has the same properties as the lysate that can be obtained by the method according to the invention is encompassed by the present invention.

The lysate described above which contains at least two different helper proteins is preferred according to the invention.

The invention also encompasses lysates containing essentially one helper protein.

A lysate according to the invention is particularly preferred in which the helper proteins are selected from the following group:

helper proteins of the DnaK system (DnaK, DnaJ and/or GrpE)

helper proteins of the GroE system (GroEL, GroES)

chaperonins

protein disulfide isomerase

trigger factor

prolyl-cis-trans isomerase

Blends of various lysates according to the invention may prove to be particularly advantageous. This enables the number of helper proteins and their concentration to be optimized for the respective in vitro translation and in vitro transcription and translation of the target protein.

A preferred embodiment is a blend comprising one or more lysates according to the invention together with a lysate containing all components that are required for an in vitro translation or for an in vitro transcription/translation.

The invention also concerns a strain which is suitable for obtaining in vitro translation lysates that has been transformed with a vector containing one or more genes coding for one or more helper proteins.

The invention also concerns the use of a lysate according to the invention or a blend according to the invention for in vitro translation or for in vitro transcription/translation. Furthermore the invention encompasses the use of a lysate according to the invention or of a blend according to the invention in a CECF or CFCF reactor. Such an experimental arrangement is embodied in the methods of continuous exchange cell-free (CECF) and continuous flow cell-free (CFCF) protein synthesis (U.S. Pat. 5,478,730; EPA 0 593 757; EPA 0 312 612; Baranov & Spirin (1993) Meth. Enzym. 217, 123-142). CECF reactors consist of at least two discrete chambers which are separated from one another by a porous membrane. The high molecular components in the reaction chamber are held back by this porous interface whereas low molecular components are exchanged between the reaction chamber and supply chamber. In the CFCF method a supply solution is pumped directly into the reaction chamber and the end products of the reaction are pressed out of the reaction compartment through one or more ultrafiltration membranes. Such reactor types have been designed for continuous exchange cell-free (CECF) and continuous flow cell-free (CFCF) protein synthesis (U.S. Pat. No. 5,478,730; EPA 0 593 757; EPA 0 312 612; Baranov & Spirin (1993) Meth. Enzym. 217, 123-142).

Surprisingly the addition of helper proteins also considerably increased the expression rate of the target proteins.

According to the invention the target proteins can be all types of prokaryotic and eukaryotic proteins and also archaeal proteins. A particular problem with previous in vitro transcription/translation systems was the expression of secretory proteins and membrane proteins especially when folding helper proteins were not present in adequate quantities. Although a successful expression of lipoproteins and membrane-bound proteins has been described in the prior art, this expression is subject to substantial limitations (Hupa and Ploegh, 1997; Falk et al., 1997). The method according to the invention can be particularly suitable for the expression of lipoproteins and membrane-bound proteins and secretory proteins as target proteins since folding helper proteins can be provided in adequate amounts by means of coexpression.

Furthermore, a surprising advantage turned out to be the fact that active telomerase can be produced by adding the lysates according to the invention to a cell-free in vivo translation system. It has not previously been possible to express active telomerase, neither in prokaryotic cells nor in cell-free prokaryotic lysates. Thus the present invention also concerns the use of a lysate according to the invention or a blend according to the invention for the in vitro translation or for the in vitro transcription/translation of telomerase. The cell-free in vitro expression of telomerase using prokaryotic lysates was achieved according to the invention by adding a lysate according to the invention containing the helper proteins DnaK and DnaJ to an E. coli extract prepared in a conventional manner. The addition of this lysate according to the invention prevents the aggregation of the unfolded catalytic subunit of telomerase and enables its reconstitution with the RNA component hTR to form active telomerase. Hence the present invention provides for the first time a method for the cost-effective production of active and pure telomerase.

Since telomerase is expressed in all eukaryotes ranging from yeast to humans, the analysis of “pure” telomerase is difficult since cofactors from the expression systems are basically always additionally present.

Since most post-translational modifications of eukaryotic cells are not present in E. coli, it is now possible to for example specifically modify in vitro synthesized telomerase with kinases and thus investigate the mode of action and effects of these modifications.

Previously there were also constraints on the introduction of unnatural amino acids in cellular expression systems for structural and functional analyses or for specific post-translational modifications (e.g. phosphorylations) (Liu J.-P. (1999) Faseb J. 13, 2091). The cell-free synthesis of telomerase now for example enables unnatural amino acids to be incorporated using all possible methods such as the incorporation of ¹⁵N- or ¹³C-labelled amino acids for NMR investigations, seleno-labelled amino acids for X-ray crystallographic analysis, fluorescent-labelled or spin-labelled amino acids (Hohsaka et al., (1999) J. Am. Chem. Soc. 121, 12194) for examining binding mechanisms.

### BRIEF DESCRIPTION OF THE DRAWINGS

FIG. 1: Telomerase fraction in the pellet and in the supernatant of the centrifugate of the reaction products obtained with and without addition of helper proteins.

FIG. 2: Proportion of a dissolved fusion protein as a function of the amount of added helper proteins.

FIG. 3: Proportion of soluble telomerase in E. coli lysates from two different preparations in a liquid or lyophilized state with and without addition of helper proteins.

FIG. 4: Influence of the addition of individual helper proteins to the lysate on the amount of soluble telomerase.

FIG. 5: Influence of the addition of DnaK and DnaJ with and without GrpE on the amount of soluble telomerase.

FIG. 6: Influence of helper proteins of the DnaK system on the activity of the green fluorescent protein (GFP).

FIG. 7: Increase in the amount of synthesized telomerase as a function of the addition of helper proteins.

FIG. 8: Effect of using lysates from cells transformed with the DnaJ/DnaJ/GrpE system on the proportion of soluble telomerase.

FIG. 9: Proportion of soluble telomerase in lysates from the non-transformed A19 strain which were mixed with 25% or 50% lysate from the A19 strain transformed with a plasmid coding for proteins from the DnaK system.

FIG. 10: Coomassie stained SDS gel of a cell-free expression of rhodanese (35 kDa); lane 1 and lane 2: without addition of RTS GroEL/ES lysate, lane 3 and lane 4: with addition of RTS GroEL/ES lysate. The respective supernatant fractions are applied in lanes 1 and 3 and the precipitate fractions are applied in lanes 2 and 4.

FIG. 11: Total activity of the cell-free expressed rhodanese as a function of lysate containing the helper protein; column 1: expression without addition of GroEL/ES lysate; column:2 expression with addition of GroEL/ES lysate. The vention is further elucidated by the following examples.

### A. Reaction Components Used

1. Plasmids

pIVEX2.3-GFP: The gene for the green fluorescent protein from Aequoria victoria (Prasher et al. (1992) Gene 111, 229) was cloned into the pIVEX2.3 vector (Roche Diagnostics GmbH Mannheim, Germany) by means of the NcoI cleavage site.

pIVEX2.4 b-Mal-Epo: The gene for the maltose binding protein was isolated from pMAL-p2 (New England Biolabs, Beverley, Mass., USA) and cloned into the vector pIVEX2.4 b. The gene for human erythropoietin (Jacobs et al. (1985) Nature 313, 806) without the signal sequence was cloned behind this gene to form pIVEX2.4b-Mal-Epo.

pIVEX2.4bNde-hTERT: The gene for the catalytic subunit of human telomerase (Autexier C. et al. (1996) EMBO Journal, 15, 5928) was cloned into the pIVEX2.4bNde vector by means of the Nde I cleavage site to form pIVEX2.4bNde-hTERT.

pIVEX2.4-rhodanese: The bovine mitochondrial rhodanese gene (Miller D.M. et al. (1991), J. Biol. Chem. 266, 4686) was cloned into the pIVEX2.4 vector by means of the Nco I cleavage site to form pIVEX2.4-rhodanese.

2. Helper Protein Plasmids

pRDKJG which codes for the proteins DnaK, DnaJ and GrpE (Dale GE et al. (1994) Protein Eng, 7, 925) and purified DnaK, DnaJ and GrpE protein were obtained from Dr. J. Schönfeld Hoffmann-La Roche Ltd., Basle, Switzerland.

pREP4-groESL which codes for the proteins Gro-EL and Gro-ES was obtained from P. Caspers (Caspers et al. (1994) Cell Mol. Biol. 40, 635-44). Purified GroEL and GroES proteins were obtained from Dr. H. Schönfeld Hoffmann-La Roche Ltd., Basle, Switzerland.

3. E. coli S30 Lysate

The lysate was prepared using an E. coli A19 strain according to the method of Zubay (Annu. Rev. Genet. (1973) 7, 267).

### SPECIFIC EMBODIMENTS

### Example 1a

### Influence of Helper Proteins on the Solubility of Telomerase

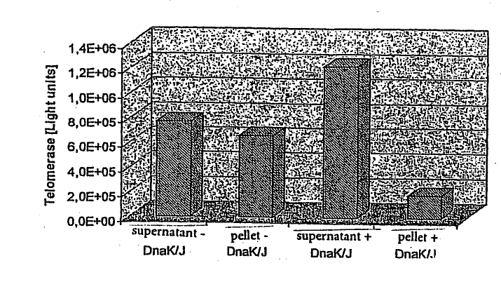

The pIVEX2.4bNde-hTERT plasmid was used in the bacterial in vitro expression system with and without the addition of 1 μM of each of the helper proteins DnaK, DnaJ and GrpE. The Rapid Translation System RTS 500 E. coli circular template Kit (Roche Diagnostics GmbH) was used for the expression. The helper proteins were used in a purified form. The reaction products were subsequently centrifuged for 2 min at 10,000×g. The resulting pellet and the supernatant were taken up in SDS sample buffer and applied to an SDS gel. The SDS gel was analysed by means of Western blot. The amounts of detected protein are shown in FIG. 1.

Result: A substantially higher proportion of dissolved telomerase (supernatant fraction) was present when helper proteins were added.

### Example 1b

### Influence of Helper Proteins of the DnaK System on the Solubility of a Fusion Protein Consisting of Maltose Binding Protein and Erythropoietin

The fusion protein was synthesized by the expression vector pIVEX2.4b-Mal-Epo in the bacterial in vitro expression system with and without addition of 1 μM of each of the helper proteins DnaK, GrpE and DnaJ (analogously to example 1a). The reaction products were subsequently centrifuged for 2 min at 10,000×g. The resulting pellet and the supernatant were taken up in SDS sample buffer and applied to an SDS gel. The SDS gel was analysed by means of Western blot.

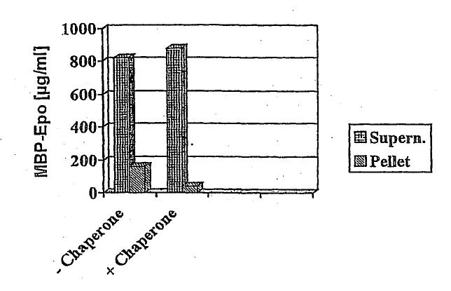

Result: An increasing proportion of dissolved fusion protein (supernatant fraction =supernatant) is present when increasing amounts of helper proteins are added (FIG. 2).

### Example 2

### Influence of Helper Proteins in Various Lysate Preparations and Lysate Lyophilisates.

As in example 1, E. coli lysates from 2 different preparations in a liquid or lyophilized state were used in a telomerase expression with and without addition of 1 μM of each of the helper proteins DnaK, DnaJ and GrpE.

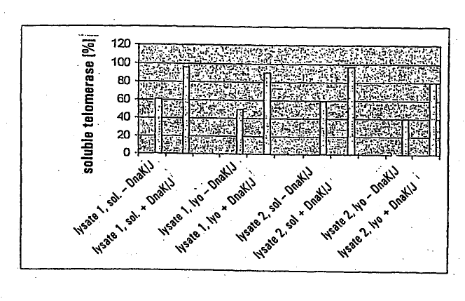

Result: A similar positive effect was found for the helper protein substitution in both lysate preparations. The lyophilized lysate exhibited a lower proportion of soluble telomerase. Also in this case the addition of helper protein increased the solubility (FIG. 3).

### Example 3

### Addition of Individual Helper Proteins to the Lysate

In this example only the purified individual components at a concentration of 1 μM were added and not the entire DnaK system comprising DnaK, DnaJ and GrpE. The analysis was as in Example 1.

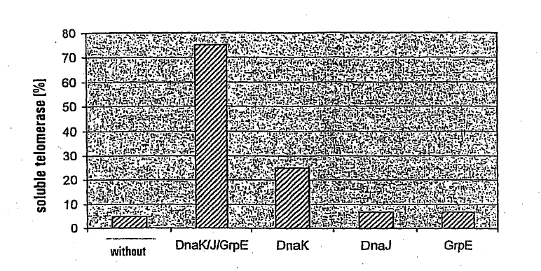

Result: DnaJ and GrpE did not have a positive effect on the solubility of telomerase, whereas DnaK had a slight positive effect that was, however, reproducible (FIG. 4).

### Example 4

### A Mixture of DnaK and DnaJ is Sufficient

A mixture of DnaK and DnaJ with and without addition of GrpE was tested as in Example 1.

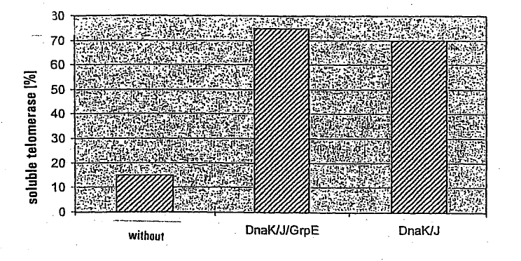

Result: The mixture of DnaK and DnaJ had the same effect as the total mixture of all 3 components (FIG. 5).

### Example 5

### Influence of Helper Proteins of the DnaK System on the Activity of the Green Fluorescent Protein (GFP)

Wild-type GFP was expressed similarly to Example 4 without the oxygen required for folding by filling the reaction vessel from the RTS 500 kit to the top. After completion of the reaction the reaction product was pipetted into an open vessel and stored for 24 hours in a refrigerator in the presence of atmospheric oxygen. During this period the correctly folded fraction of the GFP protein can oxidize and thus form the fluorophore. The activity of the GFP protein was then measured on the basis of the fluorescence.

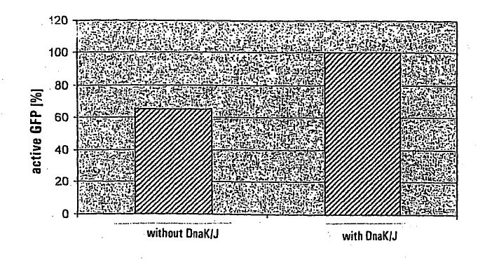

Result: The activity of GFP is increased by adding a mixture of DnaK and DnaJ (FIG. 6).

### Example 6

### Increase in the Amount of Synthesized Telomerase as a Function of the Addition of Helper Proteins

Similarly to Example 4, 1 μM, 2 μM and 3 μM amounts of the two helper proteins DnaK and DnaJ were used in the telomerase expression. However, the reaction products were then centrifuged for 30 minutes at 100,000×g and the fractions were analysed in a Western blot.

Result: Whereas 40% insoluble telomerase was still present with 1 μM of the mixture, the proportion of insoluble telomerase was reduced to 8% with 2 μM of the mixture and to <1% with 3 μM.

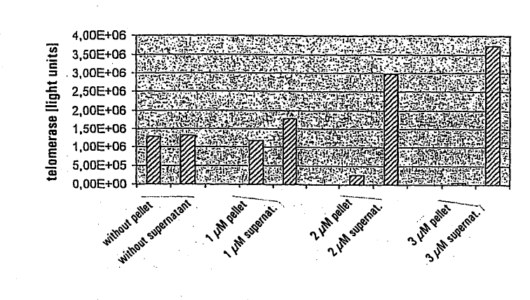

The total amount of synthesized telomerase increased considerably in all mixtures containing helper protein. In the mixture containing 3 μM DnaK/DnaJ the increase was even more than 50% (FIG. 7).

### Example 7

### Effect of Helper Proteins on the Measured Activity of Reconstituted Telomerase

Telomerase was expressed in the presence of 0 μM, 2 μM and 10 μM each of DnaK and DnaJ. Subsequently the mixtures were reconstituted with the RNA component and an activity test was set up using the Telo TAGGGG telomerase PCR ELISA (Roche Diagnostics GmbH). The telomerase was reconstituted according to the procedure of Weinrich S. L. et al. (1997) Nature Genet, 17, 498.

Before use in the in vitro protein synthesis, the helper proteins were firstly heat-treated for 30 min at 70° C. as a negative control.

TABLE 1

| | μ M | | |
|---|---|---|---|
| | Chaperone DnaK/DnaJ | | |
| relative telomerase activity [absorption units] | 0 μM | 2 μM | 10 μM |
| telomerase activity with active chaperones | 0.003 | 0.04 | 0.05 |
| telomerase activity with heat-inactivated | 0.003 | 0.003 | 0.003 |
| chaperones | | | |

Result: With helper proteins the activity was increased by more than 10-fold compared to the mixture without helper proteins. In contrast the heat-treated helper proteins were completely inactive.

### Example 8

### Production of Strains Producing Helper Protein

The strain A19 and the strain X1-blue were transformed with plasmids (see under A) which either contained the helper proteins from the DnaK/DnaJ/GrpE system or from the GroEL/ES system behind an IPTG-inducible promoter. The strain A19 has a mutation in the Rnase I gene whereas the XI-Blue strain has a deficiency in protease genes.

The transformed cells were cultured on LB medium and induced for 30 min with IPTG (final concentration up to 1 mM) at an optical density of 1.0 measured at a wavelength of 600 nm.

A lysate was prepared from these bacteria for the in vitro translation corresponding to the procedure of Zubay G (1973) Annu. Rev. Genet. 7, 267. After separating the lysates on SDS gels and staining with Coomassie Brilliant Blue, it was shown that all transformed strains expressed the corresponding proteins from the DnaK/DnaJ/GrpE system or the GroEL/ES system.

### Example 9a

### Use of Lysates From Cells Transformed With the DnaK/DnaJ/GrpE System

The lysates from cells transformed with the DnaK/DnaJ/GrpE system were subsequently used for in vitro translation with the telomerase gene. The lysates from the untransformed strains were used as a comparison.

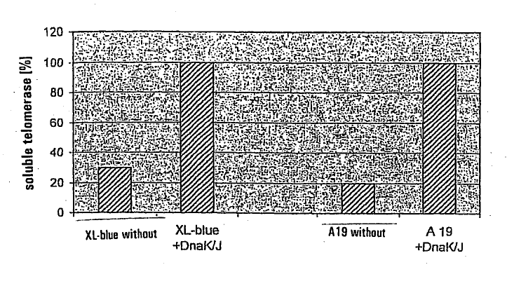

It was shown that telomerase that was 100% soluble was expressed using the lysate from the IPTG-induced transformed strains in contrast to the untransformed strains (FIG. 8).

### Example 9b

It was shown that telomerase that was 100% soluble was expressed using the lysate from the IPTG-induced transformed strains in contrast to the untransformed strains.

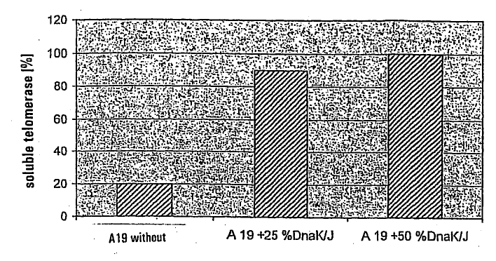

Result: Even the lysate containing only 25% of the helper protein was sufficient to increase the solubility of telomerase to 90%. Completely soluble telomerase was formed using 50% of this lysate (FIG. 9).

### Example 9c

### Use of Lysates Prepared from Cells which were Transformed with pREP4-groESL

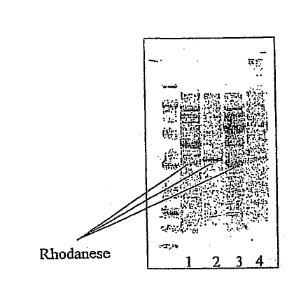

Bovine rhodanese was expressed in a bacterial in vitro expression system (Rapid Translation System RTS 500 E. coli HY Kit, Roche Diagnostics GmbH) using the pIVEX2.4-rhodanese plasmid (24 h, 30° C.) where the expression was carried out once without addition of transformed lysate (conditions as stated in the product description of the manufacturer) and in the other case with addition of 50% of a lysate (from cells which had been transformed with pREP4-groESL plasmid and had overexpressed GroEL and GroES by incubation). The reaction mixtures were subsequently centrifuged for 5 min at 10,000×g, the resulting pellet and the supernatant was taken up in SDS sample buffer, separated on an SDS gel and stained with Coomassie blue (see FIG. 10).

Result: Already 50% of the lysate containing helper proteins was sufficient to increase the solubility of rhodanese to 90%.

Subsequently it was examined whether the use of the lysate containing GroEL/ES was also able to improve the activity of the expressed rhodanese in addition to improving the solubility and hence the enzyme activity of the two preceding reactions was determined by the method of Weber, F. and Hager-Hartl, M. (Methods Mol. Biol. (2000), 140, 117).

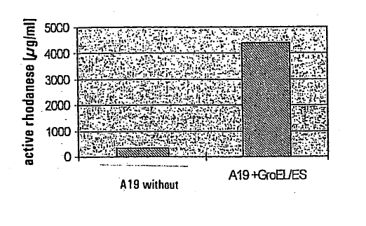

Result: The activity of rhodanese was also significantly increased by adding the lysate containing the helper protein (GroEL/ES)(see FIG. 11).

What is claimed is:

1. A method for producing a lysate containing helper proteins comprising
(a) transforming a strain which is suitable for obtaining in vitro translation lysates with a vector comprising one or more genes coding for one or more helper proteins,
(b) expressing the helper proteins in this strain, and
(c) obtaining the lysate containing helper proteins from these strains.

2. The method of claim 1, characterized in that the strain was transformed with various vectors which differed at least in that the genes contained therein code for different helper proteins.

3. The method of claim 1, wherein the strain has at least one of the following properties: low content or deficiency of RNAse, low content or deficiency of exonuclease, low content or deficiency of protease.

4. The method of claim 1, wherein the lysate is obtained in such a manner that additionally all components are present in the lysate which are required for an in vitro translation or for an in vitro transcription/translation.

5. A lysate containing helper proteins obtainable by the method of claim 1.

6. The lysate of claim 5, wherein it contains at least two different helper proteins.

7. The lysate of claim 5 containing essentially one helper protein.

8. The lysate of claim 5, wherein the helper proteins are selected from the group consisting of helper proteins of the DnaK system (DnaK, DnaJ and/or GrpE), helper proteins of the GroE system (GroEL, GroES), chaperoning, protein disulfide isomerase, trigger factor, and prolyl-cis-trans isomerase.

9. A blend of various lysates as claimed in claim 7.

10. A blend of one or more lysates as claimed in claim 5 with a lysate containing all components that are necessary for an in vitro translation or for an in vitro transcription/translation.

11. A strain which is suitable for obtaining in vitro translation lysates which has been transformed with a vector containing one or more genes coding for one or more helper proteins.

12. Use of a lysate as claimed in one of the claims 5 to 8 or of a blend as claimed in one of the claims 9 or 10 for in vitro translation or for in vitro transcription/translation.

13. Use of a lysate as claimed in one of the claims 5 to 8 or of a blend as claimed in one of the claims 9 or 10 for in vitro translation or in vitro transcription of telomerase.

14. Use of a lysate as claimed in one of the claims 5 to 8 or of a blend as claimed in one of the claims 9 or 10 in a CECF or CFCF reactor.
